(19) United States
(12) Patent Application Publication
(10) Pub. No.: US 2024/0011958 A1
(43) Pub. Date: Jan. 11, 2024

(54) PORTABLE GAS SENSING DEVICE

(71) Applicants: Gus Hammond, Chicago, IL (US); Ahmad Hamoud, Chicago, IL (US)

(72) Inventors: Gus Hammond, Chicago, IL (US); Ahmad Hamoud, Chicago, IL (US)

(21) Appl. No.: 17/857,806

(22) Filed: Jul. 5, 2022

(51) Int. Cl. G01N 33/00; H02J 7/00; H02J 50/10; G08B 21/14; G08B 21/18

(52) U.S. Cl. CPC G01N 33/0009; H02J 7/0063; H02J 7/00034; H02J 7/0068; H02J 7/0044; H02J 50/10; G01N 33/004; G08B 21/14; G08B 21/182

(57) ABSTRACT

A gas sensing device adapted to be carried by a person includes a housing, a gas sensor arranged, at least partially, inside the housing and configured to detect a gas in a vicinity of the device, an indicator mounted to the housing and configured to indicate a level of the concentration of the gas in the vicinity of the device, and a controller arranged in communication with the gas sensor and the indicator. The controller is configured to determine the level of concentration of the gas in the vicinity based on inputs received from the gas sensor, and actuate the indicator based on the detected level of concentration of the gas in the vicinity of the gas.

### TECHNICAL FIELD

The present disclosure relates, generally, to a gas sensing device, and more particularly relates to a portable gas sensing device suitable to be carried by a person and adapted to detect a concentration of a gas, for example, carbon monoxide in the ambient.

### BACKGROUND

Carbon monoxide is a non-desirable gas especially when present in high concentrations. Exposure to excess concentration of carbon monoxide in the ambient or a closed space may create difficulty in breathing and other health concerns to humans. Prolonged exposure to carbon monoxide even may lead to many diseases and long-term problems. While gas sensors are, generally, installed to detect the carbon monoxide in the ambient, these sensors are prone to failure due to a lack of timely and adequate maintenance. In addition, these sensors are unable to provide individual with adequate information about the concentration of the gas or the exposure the individual is having to the gas.

### SUMMARY

Embodiments of the disclosure provide a gas sensing device adapted to be carried by a person. The gas sensing device includes a housing, a gas sensor arranged, at least partially, inside the housing and configured to detect a gas in a vicinity of the device, an indicator mounted to the housing and configured to indicate a level of the concentration of the gas in the vicinity of the device, and a controller arranged in communication with the gas sensor and the indicator. The controller is configured to determine the level of concentration of the gas in the vicinity based on inputs received from the gas sensor, and actuate the indicator based on the detected level of concentration of the gas in the vicinity of the gas.

In some embodiments, the gas sensor is a carbon monoxide sensor for detecting a carbon monoxide in the vicinity of device.

In some embodiments, the device further includes a temperature sensor to facilitate measuring of a temperature in the vicinity.

In some embodiments, the controller determines the level of concentration based on the detected temperature.

In some embodiments, the indicator is a light emitting diode display configured to emit one or more lights of one or more colors based on the level of concentration of the gas in the vicinity of the device.

In some embodiments, the light emitting diode display emits a first light of a first color in response to the level of concentration of the gas being below a first threshold value.

In some embodiments, the light emitting diode display emits a light of a second color in response to the level of concentration of the gas being between a first threshold value and a second threshold value, wherein the second threshold value being greater than first threshold value.

In some embodiments, the light emitting diode display emits a light of a third color when the level of concentration of the gas is between the second threshold value and a third threshold value, the third threshold value being greater than first threshold value.

In some embodiments, the light emitting diode display emits a light of a fourth color when the level of concentration of the gas is between the third threshold value and a fourth threshold value, the fourth threshold value being greater than third threshold value.

In some embodiments, the light emitting diode display emits a light of a fifth color when the level of concentration of the gas is above the fourth threshold value.

In some embodiments, the device further includes a battery arranged inside the housing to provide electrical power the gas sensor, the indicator, and the controller.

In some embodiments, the device further includes at least one charging socket to enable a charging of the battery.

In some embodiments, the device further includes a charging socket to receive electrical power from a mobile phone.

In some embodiments, the device further includes a transceiver to facilitate a communication between the device and an external device.

In some embodiments, the transceiver is a short range transceiver.

In some embodiments, the device further includes at least one charging connector to enable a charging of an external device.

In some embodiments, the device further includes a switch to turn on and turn off the device.

In some embodiments, the housing includes a shape of sleeve of a mobile phone to hold the mobile phone.

In some embodiments, the device further includes a wireless charging coil to facilitate a wireless charging of an external device.

### BRIEF DESCRIPTION OF THE ACCOMPANYING DRAWINGS

Having thus described example embodiments of the present disclosure in general terms, reference will now be made to the accompanying drawings, which are not necessarily drawn to scale, and wherein:

FIG. 1 illustrates a gas sensing device suitable to be carried by a person, according to an embodiment of the disclosure;

FIG. 2 illustrates a block diagram of the gas sensing device of FIG. 1 depicting various components of the gas sensing device arranged inside a housing of the gas sensing device, according to an embodiment of the disclosure;

FIG. 3 illustrates a gas sensing device having a housing in a shape of phone sleeve to hold a phone, according to an embodiment of the disclosure; and

FIG. 4 illustrates a gas sensing device affixed to a mobile phone, according to an embodiment of the disclosure.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, apparatus and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. The use of any term should not be taken to limit the spirit and scope of embodiments of the present invention.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting.

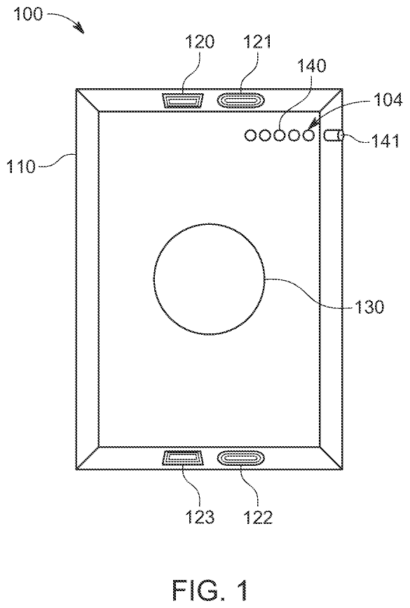

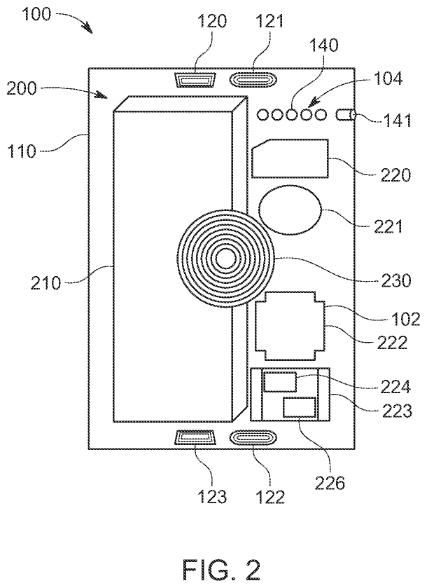

FIGS. 1 and 2 shows a gas sensing device 100 (hereinafter referred to as device 100) suitable to be carried by a person and configured to detect a presence and concentration of a gas, for example, carbon monoxide, in a vicinity of the person is shown. The device 100 includes a housing 110 defining a chamber 200 to house/hold/support various components of the device 100. The device 100 includes a gas sensor 102, for example, carbon monoxide sensor 222, to detect the gas, for example, carbon monoxide, in the vicinity of the device 100, and a temperature sensor 221 to monitor and determine a temperature of the ambient in the vicinity of the device 100. Although the gas sensor 102 is contemplated as the carbon monoxide sensor 222, it may be envisioned that the gas sensor 102 may be adapted to detect any other gas, for example, chlorine, fluorine, or any other gas known in the art.

Further, the device 100 may include an indicator 104 suitable to indicate a level of the concentration of the gas detected by the gas sensor in the vicinity of the device 100. In the illustrated embodiment, the indicator 104 is shown as LED display 140 to visually indicate the level of carbon monoxide in the surrounding of the device 100. In the illustrated embodiment, the LED display 140 is a multi-color LED display configured to display lights of distinct colors depending upon the level of concentration the gas, for example, the carbon monoxide, in the surrounding of the device 100. For example, the LED display 140 is configured to display a light of a first color, for example, blue color, when the level of concentration of the gas in the surrounding of the device 100 is below a first threshold value. The first threshold value may correspond to a safest value. Additionally, the LED display 140 is configured to display lights of distinct colors, for example, green, yellow, orange, and red depending on the levels of the concentration of the gas lying above the first threshold value. For example, the LED display 140 may display a light of a second color, for example, a green color, when the level of the concentration of the gas in the surrounding area is between the first threshold value and a second threshold value. Similarly, the LED display 140 may a display a light of a third color, for example, a yellow light, when the level of the concentration of the gas in the surrounding area is between the second threshold value and a third threshold value. Moreover, the LED display 140 may display a light of a fourth color, for example, an orange light, when the level of the concentration of the gas in the surrounding area is between the third threshold value and a fourth threshold value. Further, the LED display 140 is configured to display a light of a fifth color, for example, red color, when the level of concentration of the gas in the surrounding of the device 100 is above the fourth threshold value. The fourth threshold value may correspond to a most dangerous level of the gas in the surrounding area. It may be appreciated that the second threshold value is greater than the first threshold value, but less than the third threshold. Similarly, the third threshold value is less than the fourth threshold value, but greater than the second threshold value. Although, the LED display 140 display 5 different lights, it may be appreciated that the LED display 140 may be configured to display any number of lights depending upon the level of the concentration of the gas detected by the gas sensor 102. For example, in some embodiments, the LED display may include only the red light. In such a case, the light is not illuminated when the concentration of the gas in the vicinity of the system is below an associated threshold value.

Although the indicator 104 is contemplated as a visual indicator, it may be appreciated that the indicator 104 may be an audio indicator. In such a case, the indicator 104 is actuated and emit a sound when the concentration of the gas, detected by the gas sensor 102, is above a threshold value. Similarly, the indicator 104 may provide vibrator, and generates vibrations when the detected concentration of the gas is above a threshold value, providing a vibrotactile feedback to the person.

To power the indicator 104, the gas sensor 102, the temperature sensor 221, and other electrical and electronic components of the device 100, the device 100 may include at least one battery 210 arranged inside the housing 110. In the illustrated embodiment, the battery 210 is a rechargeable battery, however a non-rechargeable battery may also be contemplated. In a case where the battery 210 is non-rechargeable battery, the battery 210 may be easily replaced with a new battery upon discharge of the battery 210. Further, to facilitate a charging of the battery 210, the device 100 may include at least one charging socket, for example, a first charging socket 120 of USB-C type and a second charging socket 121 of USB-A type, that enables the charging of the at least one battery 210 from an external power source via a suitable charging cable. Although two charging sockets 120, 121 are contemplated, it may be appreciated that the device 100 may include a single charging socket or more than two charging sockets.

Further, the device 100 includes a switch 141 that is ON-OFF switch to facilitate turning on and turning off of the device 100. As shown, the switch 141 is connected to the housing 110 and extends outwardly of the housing 110, and may be positioned proximate to an edge of the housing 110. In an embodiment, the user may press the switch 141 to turn on and turn off the device 100. When the switch 141 is turned on, the electric power from the battery 210 is supplied to the electrical components of the device 100 to enable a working of the device 100 and measure the level of the concentration of the gas in the surrounding of the device 100. While the electric power to the electrical components of the device 100 from the battery 210 is interrupted when the switch 141 is turned off, stopping the working of the device 100. Moreover, the device 100 may include a wireless charging coil 230 arranged inside the housing 130 and disposed in electrical communication with the battery 210 to enable a wireless charging of an external device, for example, a smart phone of the person carrying the device 100. In an embodiment, to enable the wireless charging of the external device, the external device may be positioned on a surface 130 of the housing 110.

Further, the device 100 may include at least one charging connectors, for example, a first charging connector 122 of USB-C type and a second charging connector 123 of USB-A type, electrically coupled to the battery 210 and configured to facilitate a charging of an external device, for example, a mobile phone. In an embodiment, the charging connectors 122, 123 may be charging sockets extending inside the housing 110 from an outer surface of the housing 110. In an embodiment, the charging connectors 122, 123 may extend outside the housing 110 and may be inserted inside a charging port of the mobile phone to enable the charging of the mobile phone. Although two charging connectors 122, 123 are contemplated, it may be appreciated that the device 100 may include a single charging connector or more than two charging connectors.

Additionally, the device 100 includes a transceiver 220, arranged at least partially inside the housing 110, to enable a communication and exchange of data between the device 100 and an external device, for example, a mobile phone of a technician, in case the device 100 is detached from the mobile phone. The mobile phone is also equipped with an application to further relay information to a remote location such as a central station. The transceiver 220 may be a short-range communication unit, for example, a Bluetooth™ transceiver.

Further, the device 100 includes a controller 223 arranged in communication with the gas sensor 102, the temperature sensor 221, the battery 210, the transceiver 220, the switch 141, the indicator 104, and other electrical and electronic components of the device 100, and is adapted to receive the data from the sensors 102, 221. In an embodiment, the controller 223 receives data about a presence and concentration of the gas in the vicinity of the device 100, and determines the concentration of the gas based on the data. Further, the controller 223 is configured to compare the detected level of concentration of the gas with one or more threshold values, and actuates the indicator 104 based on the comparison. For example, the controller 223 may actuate the LED (light emitting diode) display 140 such that the display 140 shows the blue light if the level of concentration of gas determined by the controller 223 is below the first threshold value. Similarly, the controller 223 may actuate the LED display 140 such that the display 140 shows green light, yellow light, orange light, or the red light depending on the level of the concentration of the gas determined by the controller 223.

In an embodiment, the controller 223 is configured to record/store a date and time stamp when the detected concentration of the gas is above a certain threshold value, for example, the fourth threshold value, or above the third threshold value. Also, the controller 223 may store the value of the concentration of the gas when the detected concentration of the gas is above fourth threshold value or the third threshold value.

In an embodiment, the controller 223 may monitor and store the location coordinates of the device 100 in real time. In an embodiment, the controller 223 may store the location coordinates of the location where the level of concentration of the gas is above the first threshold value, and also stores the associated value of the concentration of the gas. In an embodiment, the device 100 may include a display that may indicate a remaining capacity of the battery 210. Also, the controller 223 is in communication with the mobile device the person or the central station via the transceiver 220 and share all the data monitored and collected by the controller 223 to the mobile device or the central station.

The controller 223 may include a processor 224 for executing specified instructions, which controls and monitors various functions associated with device 100. The processor 224 may be operatively connected to a memory 226 for storing instructions related to the functioning of the device 100. The memory 226 as illustrated is integrated into the controller 223, but those skilled in the art will understand that the memory 226 may be separate from the controller 223 but onboard the device 100, and/or remote from the controller 223 and the device 100, while still being associated with and accessible by the controller 223 to store information in and retrieve information from the memory 226 as necessary during the operation of device. Although the processor 224 is shown, it is also possible and contemplated to use other electronic components such as a microcontroller, an application specific integrated circuit (ASIC) chip, or any other integrated circuit device. Moreover, the controller 223 may refer collectively to multiple control and processing devices across which the functionality of the device 100 may be distributed. For example, the indicator 104, the transceiver 220, the gas sensor 102, the temperature sensor 221, may each have one or more controllers that communicate with the controller 223.

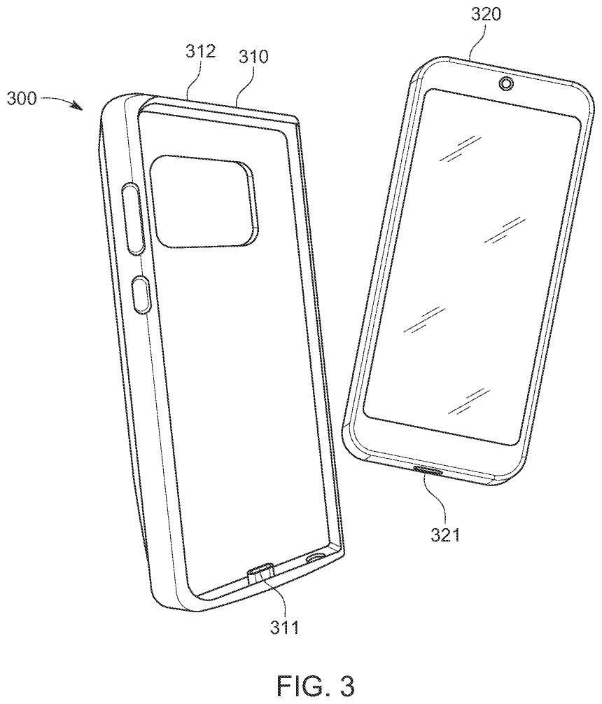

Referring to FIG. 3, a gas sensing device 300 is shown according to an alternative embodiment of the disclosure. The device 300 is similar to the device 100 except that a housing 310 of the device 300 includes a shape of a sleeve 312 of a mobile phone 320 to hold the mobile phone 320. Moreover, a charging connector 311 of the device 300 extends outwardly of a sidewall 322 of the housing 310 such that the charging connector 311 extends inside a charging socket 321 of the mobile phone 320 when the mobile phone 320 is inserted inside the sleeve 312.

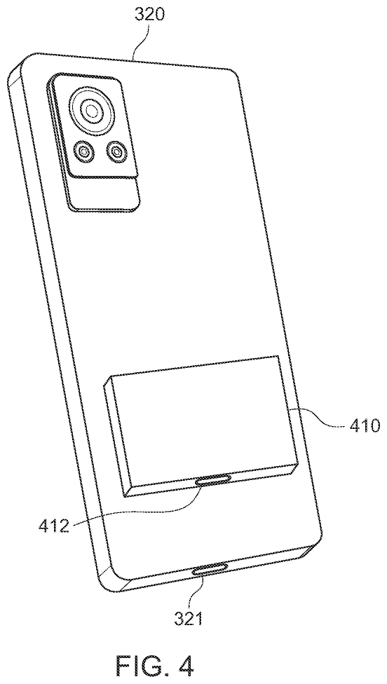

Referring to FIG. 4, a gas sensing device 400 is shown, according to yet another embodiment of the disclosure. The device 400 is similar to the device 100 except that the battery 210 is omitted from the device 400. Accordingly, the device 400 is suitable for attachment to the mobile phone 320 and receives power from the phone 320 via a suitable USB cable that can be attached with the USB port 321 of the phone 320 and a charging socket 412 the device 400.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A gas sensing device adapted to be carried by a person, the gas sensing device comprising:
a housing;
a gas sensor arranged, at least partially, inside the housing and configured to detect a gas in a vicinity of the device;
an indicator mounted to the housing and configured to indicate a level of the concentration of the gas in the vicinity of the device; and
a controller arranged in communication with the gas sensor, and the indicator, and configured to
determine the level of concentration of the gas in the vicinity based on inputs received from the gas sensor, and
actuate the indicator based on the detected level of concentration of the gas in the vicinity of the gas.

2. The device of claim 1, wherein the gas sensor is a carbon monoxide sensor for detecting a carbon monoxide in the vicinity of device.

3. The device of claim 1 further including a temperature sensor to facilitate measuring of a temperature in the vicinity.

4. The device of claim 3, wherein the controller determines the level of concentration based on the detected temperature.

5. The device of claim 1, wherein the indicator is a light emitting diode display configured to emit one or more lights of one or more colors based on the level of concentration of the gas in the vicinity of the device.

6. The device of claim 5, wherein the light emitting diode display emits a first light of a first color in response to the level of concentration of the gas being below a first threshold value.

7. The device of claim 6, wherein the light emitting diode display emits a light of a second color when the level of concentration of the gas is between the first threshold value and a second threshold value, the second threshold value being greater than first threshold value.

8. The device of claim 7, wherein the light emitting diode display emits a light of a third color when the level of concentration of the gas is between the second threshold value and a third threshold value, the third threshold value being greater than second threshold value.

9. The device of claim 8, wherein the light emitting diode display emits a light of a fourth color when the level of concentration of the gas is between the third threshold value and a fourth threshold value, the fourth threshold value being greater than the third threshold value.

10. The device of claim 9, wherein the light emitting diode display emits a light of a fifth color in response to the level of concentration of the gas being above the fourth threshold value.

11. The device of claim 1 further including a battery arranged inside the housing to provide electrical power the gas sensor, the indicator, and the controller.

12. The device of claim 11 further including at least one charging socket to enable a charging of the battery.

13. The device of claim 1 further including a charging socket to share electrical power and exchanged data with a mobile phone.

14. The device of claim 1 further including a transceiver to facilitate a communication between the device and an external device.

15. The device of claim 14, wherein the transceiver is a short range transceiver.

16. The device of claim 1 further including at least one charging connector to enable a charging of an external device.

17. The device of claim 1 further including a switch to turn on and turn off the device.

18. The device of claim 1, wherein the housing includes a shape of sleeve of a mobile phone to hold the mobile phone.

19. The device of claim 1 further including a wireless charging coil to facilitate a wireless charging of an external device.
